(19) United States
(12) Patent Application Publication
(10) Pub. No.: US 2026/0183050 A1
(43) Pub. Date: Jul. 2, 2026

(54) Electrosurgical Device Having an Adjustable Length, Methods of Operating an Electrosurgical Device, and Methods of Manufacturing an Electrosurgical Device

(71) Applicant: Stryker European Operations Limited, Cork (IE)

(72) Inventors: Micheal Burke, Cork (IE); Scott McFarland, Greenisland (IE); Laura Constance Frey, Belfast (IE); Paul Sheridan, Wexford (IE); Syed Ashad Mustufa, Cork (IE); Molly O'Mara, Lambertville, NJ (US)

(21) Appl. No.: 19/131,813

(22) PCT Filed: Nov. 28, 2023
(86) PCT No.: PCT/IB2023/000707

§ 371 Date: May 21, 2025

Related U.S. Application Data
(60) Provisional application No. 63/428,299, filed on Nov. 28, 2022.
(60) Provisional application No. 63/513,005, filed on Jul. 11, 2023.

(51) Int. Cl. A61B 18/14; A61B 17/00; A61B 18/00

(52) U.S. Cl. CPC A61B 18/1482; A61B 2017/00398; A61B 2017/00526; A61B 2017/00991; A61B 2018/00642; A61B 2018/00708

(57) ABSTRACT

An example electrosurgical device includes a handle having a proximal end and a distal end. The handle defines an interior cavity. The electrosurgical device also includes a shaft extending from the distal end of the handle. At least a portion of the shaft is in the interior cavity of the handle and the shaft is telescopically movable relative to the handle. The electrosurgical device also includes an electrosurgical electrode extending from a distal end of the shaft and a linear actuator in the interior cavity of the handle. The linear actuator is operable to axially move the shaft relative to the handle.

### CROSS-REFERENCE TO RELATED APPLICATIONS

The present application claims the benefit of priority of U.S. Provisional Patent Application No. 63/513,005 , filed Jul. 11, 2023, and U.S. Provisional Patent Application No. 63/428,299 , filed Nov. 28, 2022, the contents of which are hereby incorporated by reference in their entirety.

### FIELD

The present disclosure generally relates to electrosurgical devices and, more specifically, to electrosurgical devices having a handle and an electrosurgical electrode extending from a shaft that is movable relative to the handle.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 depicts a simplified block diagram of an electrosurgical device, according to an example.

FIG. 2A depicts a first state of operation for an implementation of the electrosurgical device shown in FIG. 1, according to an example.

FIG. 2B depicts a second state of operation for an implementation of the electrosurgical device shown in FIG. 1, according to an example.

FIG. 3 depicts an assembly of a linear actuator, a shaft, and a sensor of an electrosurgical device, according to an example.

FIG. 4A depicts a cross sectional view of an electrosurgical device, according to an example.

FIG. 4B depicts a cross sectional view of an electrosurgical device, according to an example.

FIG. 5 depicts a flowchart of a method of operation, according to an example.

FIG. 6 depicts a flowchart of a method of operation that can be used with at least the method shown in FIG. 5, according to an example.

FIG. 7 depicts a flowchart of a method of operation that can be used with at least the method shown in FIG. 6, according to an example.

FIG. 8 depicts a flowchart of a method of operation that can be used with at least the method shown in FIG. 5, according to an example.

FIG. 9 depicts a flowchart of a method of operation that can be used with at least the method shown in FIG. 8, according to an example.

FIG. 10 depicts a flowchart of a method of operation that can be used with at least the method shown in FIG. 5, according to an example.

FIG. 11 depicts a flowchart of a method of operation that can be used with at least the method shown in FIG. 5, according to an example.

FIG. 12 depicts a flowchart of a method of operation that can be used with at least the method shown in FIG. 11, according to an example.

FIG. 13 depicts a flowchart of a method of operation that can be used with at least the method shown in FIG. 5, according to an example.

FIG. 14 depicts a flowchart of a method of operation that can be used with at least the method shown in FIG. 5, according to an example.

FIG. 15 depicts a flowchart of a method of operation that can be used with at least the method shown in FIG. 14, according to an example.

FIG. 16 depicts a flowchart of a method of manufacture, according to an example.

FIG. 17 is a block diagram of an electrosurgical system, according to an example.

FIG. 18A illustrates a perspective view of the electrosurgical device of FIG. 17 1701, according to an example.

FIG. 18B illustrates a partial cross-sectional view of the electrosurgical device of FIG. 17, according to an example.

FIG. 19 depicts a flowchart of a method of operation, according to another example.

FIG. 20 depicts a flowchart of a method of manufacture, according to another example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Some electrosurgical devices include a handle and an electrosurgical electrode attached to a shaft that is extendable and retractable with respect to the handle. The position of the electrode being adjustable relative to the handle can help an operator to apply electric current to tissue at different positions and/or depths on or within a patient. In some existing electrosurgical devices, the adjustment generally requires the operator to cease surgical operations, loosen a locking nut, manually adjust the electrode position with two hands, and retighten the locking nut prior to performing further surgical operations, which can be inefficient and tedious.

The present disclosure provides an electrosurgical device that can address one or more challenges of such electrosurgical devices. Within examples, the electrosurgical device includes a handle having a proximal end and a distal end. The handle defines an interior cavity. The electrosurgical device also includes a shaft extending from the distal end of the handle. At least a portion of the shaft is in the interior cavity of the handle and the shaft is telescopically movable relative to the handle. The electrosurgical device also includes an electrosurgical electrode extending from a distal end of the shaft and a linear actuator in the interior cavity of the handle. The linear actuator is operable to axially move the shaft relative to the handle. In this arrangement, the linear actuator can provide for automated extension and retraction of the shaft, which can help to improve operational efficiencies and/or ease of use relative to electrosurgical devices that require manual extension and retraction of the shaft relative to the handle, as described above.

In some examples, the operator can extend or retract the electrosurgical electrode using the linear actuator and then enable the electrosurgical electrode, all while gripping the handle with a single hand and without removing the single hand from the handle. This can make operation of the device more efficient and free a second hand to perform other surgical tasks. Single-handed operation can allow the operator to control irrigation or a secondary device with their non-dominant hand. This activity might otherwise be carried out by a second person such as a scrub tech or a nurse. The enhanced efficiency of the device might allow the second person to carry out other activities or attend another surgery. This can help reduce the number of man hours and cost required for each operation.

In some examples, the electrosurgical device can be configured to detect collisions of the electrosurgical electrode or the shaft with tissues or other obstructions and to responsively disable movement of the shaft. This can enhance safe operation of the electrosurgical device at or near a surgical site.

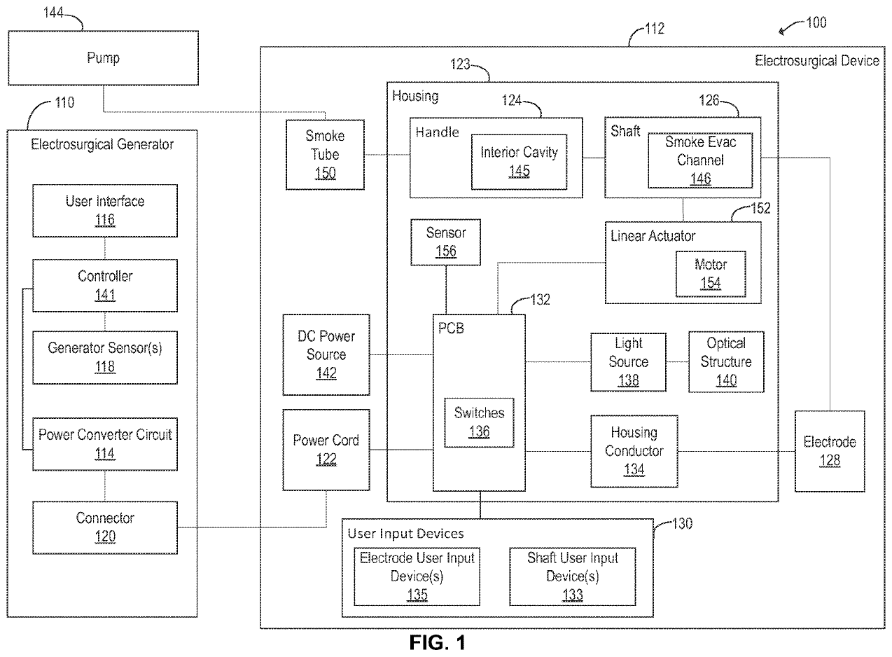

FIG. 1 is a simplified block diagram of an electrosurgical system 100, according to an example. As shown in FIG. 1, the electrosurgical system 100 includes an electrosurgical generator 110 and an electrosurgical device 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, one or more display screens, one or more indicator lights, one or more speakers, and/or one or more haptic output devices.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more generator sensor(s) 118 that can sense one or more conditions related to the electrosurgical energy and/or the target tissue. As examples, the generator sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the generator sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 kHz.

In FIG. 1, the electrosurgical generator 110 also includes a connector 120 that can facilitate coupling the electrosurgical generator 110 to the electrosurgical device 112. For example, the electrosurgical device 112 can include a power cord 122 having a plug, which can be coupled to a socket of the connector 120 of the electrosurgical generator 110. In this arrangement, the electrosurgical generator 110 can supply the electrosurgical energy to the electrosurgical device 112 via the coupling between the connector 120 of the electrosurgical generator 110 and the power cord 122 of the electrosurgical device 112.

The electrosurgical generator 110 can further include a controller 141 that can control operation of the electrosurgical generator 110. Within examples, the controller 141 can be implemented using hardware, software, and/or firmware. For instance, the controller 141 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 141, thus, can receive data and store the data in the memory as well. As shown in FIG. 1, the controller 141 can be communicatively coupled with the power converter circuit 114, the user interface 116, the generator sensor(s) 118, and/or the connector 120.

As shown in FIG. 1, the electrosurgical device 112 can include a housing 123 having a proximal end and a distal end, and an electrosurgical electrode 128 extending from the distal end of the housing 123. The housing 123 can be an elongated structure in and/or on which components of the electrosurgical device 112 can be disposed. In some examples, the housing 123 can be an integral, monolithic structure. In other examples the housing 123 can include a plurality of structures that are coupled to each other.

In FIG. 1, the housing 123 includes a handle 124 that defines an interior cavity 145, and a shaft 126 extending in a distal direction from a distal end of the handle 124. In general, the handle 124 can be configured to facilitate a user gripping and manipulating the electrosurgical device 112 while performing electrosurgery. For example, the handle 124 can have a shape and/or a size that can facilitate a user performing electrosurgery by manipulating the electrosurgical device 112 using a single hand. In one implementation, the handle 124 can have a shape and/or a size that facilitates the user holding the electrosurgical device 112 in a writing utensil gripping manner (e.g., the electrosurgical device 112 can be an electrosurgical pencil). In another implementation, the handle 124 can have a shape and/or a size that facilitates the user holding the electrosurgical device 112 in a pistol grip (e.g., the handle 124 and the shaft 126 can have longitudinal axes that are transverse relative to each other).

Additionally, for example, the handle 124 and/or the shaft 126 can be constructed from one or more materials that are electrical insulators (e.g., a plastic material). This can facilitate insulating the user from the electrosurgical energy flowing through the electrosurgical device 112 while performing the electrosurgery.

Within examples, the shaft 126 is movable relative to the handle 124. For example, the shaft 126 can be telescopically moveable relative to the handle 124. In such examples, at least a portion of the shaft 126 is in the interior cavity 145 of the handle 124, and the shaft 126 can extend in the distal direction and retract the shaft 126 in a proximal direction relative to the handle 124 (e.g., movable along a longitudinal axis of the electrosurgical device 112). As described in further detail below, the electrosurgical device 112 includes one or more features that provide for automated extension and/or retraction of the shaft 126 relative to the handle 124 responsive to a user input, and these feature(s) can help to improve operational efficiencies and/or ease of use relative to electrosurgical devices that require manual extension and retraction of the shaft 126 relative to the handle 124.

In some examples, the electrosurgical electrode 128 can be coupled to the shaft 126 and, thus, the electrosurgical electrode 128 can move together with the shaft 126 in an axial direction along the longitudinal axis relative to the handle 124. This can provide for adjusting a length of the electrosurgical device 112, which can facilitate performing electrosurgery at a plurality of different depths within tissue (e.g., due to different anatomical shapes and/or sizes of patients) and/or at a plurality of different angles. In other examples, the electrosurgical electrode 128 can be fixedly coupled to the handle 124 such that the shaft 126 is axially movable relative to both the electrosurgical electrode 128 and the handle 124. This can provide for adjusting an amount of the electrosurgical electrode 128 that is exposed at the distal end of the shaft 126.

In some implementations, the electrosurgical electrode 128 can additionally be rotatable about an axis of rotation that is parallel to the longitudinal axis of the electrosurgical device 112. In some examples, the electrosurgical electrode 128 can be rotatable relative to the handle 124 and the shaft 126. In other examples, the electrosurgical electrode 128 can be rotationally fixed relative to the shaft 126 such that the shaft 126 and the electrosurgical electrode 128 are rotatable together relative to the handle 124. Rotating the electrosurgical electrode 128 relative to the handle 124 can facilitate adjusting an angle of the electrosurgical electrode 128 relative to one or more user input device(s) 130 of the electrosurgical device 112. In this arrangement, a user can comfortably grip the handle 124 in a position in which their fingers can comfortably operate the user input device(s) 130 while the electrosurgical electrode 128 is set at a rotational position selected from among a plurality of rotational positions relative to the handle 124 based on, for example, a location, a size, and/or a shape of a surgical site in which the user is operating.

In one implementation, the electrosurgical electrode 128 can be rotatable by more than 360 degrees relative to the handle 124. This can improve an ease of use by allowing an operator to freely rotate the electrosurgical electrode 128 without limitation. However, in other implementations, the electrosurgical electrode 128 can be rotatable by less than or equal to 360 degrees (e.g., rotatable by 180 degrees, rotatable by 270 degrees, or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

Although it can be beneficial to provide for rotation of the electrosurgical electrode 128 relative to the handle 124 and/or the shaft 126, the electrosurgical electrode 128 can be rotationally fixed relative to the handle 124 and the shaft 126 in some implementations. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts).

As shown in FIG. 1, the electrosurgical device 112 can include one or more user input device(s) 130 that are operable to control operation of the electrosurgical device 112 and/or the electrosurgical generator 110. In FIG. 1, the user input device(s) 130 of the electrosurgical device 112 include at least one shaft user input device 133 and at least one electrode user input device 135. As explained in further detail below, the shaft user input device(s) 133 are operable to cause the shaft 126 to move relative to the handle 124.

The electrode user input device(s) 135 can be operable to select between the modes of operation of the electrosurgical device 112 and/or the electrosurgical generator 110. In one implementation, the electrode user input device(s) 135 can be configured to select between a cutting mode of operation and a coagulation mode of operation. Responsive to actuation of the electrode user input device(s) 135 of the electrosurgical device 112, the electrosurgical device 112 can (i) receive the electrosurgical energy with a level of power and/or a waveform corresponding to the mode of operation selected via the user input device(s) 130 and (ii) supply the electrosurgical energy to the electrosurgical electrode 128.

In FIG. 1, the electrosurgical device 112 includes a plurality of electrical components that facilitate supplying the electrosurgical energy, which the electrosurgical device 112 receives from the electrosurgical generator 110, to the electrosurgical electrode 128. For example, the electrosurgical device 112 can include at least one electrical component selected from a group of electrical components including: a printed circuit board 132 (e.g., a flexible printed circuit board) and/or one or more housing conductors 134 that can provide a circuit for conducting the electrosurgical energy from the power cord 122 to the electrosurgical electrode 128. One or more of the electrical components can be positioned in the interior cavity 145 defined by the handle 124 and/or in an inner bore defined by the shaft 126.

Within examples, the printed circuit board 132 can be implemented using hardware, software, and/or firmware. For instance, the printed circuit board 132 can include one or more processors and a non-transitory computer readable medium (e.g., volatile and/or non-volatile memory) that stores machine language instructions or other executable instructions. The instructions, when executed by the one or more processors, cause the electrosurgical device 112 to carry out the various operations described herein. The printed circuit board 132, thus, can receive data and store the data in the memory as well.

Within examples, the electrode user input device(s) 135 can include one or more buttons on an exterior surface of the handle 124. Each button of the electrode user input device(s) 135 can be operable to actuate a respective one of a plurality of switches 136 of the printed circuit board 132. In general, the switches 136 and/or the printed circuit board 132 are operable to control a supply of the electrosurgical energy from the electrosurgical generator 110 to the electrosurgical electrode 128. For instance, in one implementation, when each button is operated (e.g., depressed), the respective switch 136 associated with the button can be actuated to cause the printed circuit board 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 136 associated with the button can close the switch 136 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the printed circuit board 132 can be omitted.

In both example implementations, the electrosurgical energy supplied by the electrosurgical generator 110 can be supplied from (i) the power cord 122, the printed circuit board 132, and/or the switch(es) 136 to (ii) the electrosurgical electrode 128 by the housing conductor(s) 134. As such, as shown in FIG. 1, the printed circuit board 132 can be coupled to the power cord 122, the printed circuit board 132 can be coupled to the housing conductor(s) 134, and the housing conductor(s) 134 can be coupled to the electrosurgical electrode 128. In this arrangement, the housing conductor(s) 134 can conduct the electrosurgical energy to the electrosurgical electrode 128. The switch(es) 136 can be coupled to the printed circuit board 132 in some examples.

In general, the housing conductor(s) 134 can each include one or more electrically conductive elements that provide an electrically conductive bus for supplying the electrosurgical energy to the electrosurgical electrode 128. In some examples, the electrical components of the electrosurgical device 112 can be electrically coupled to each other in a manner that is suitable to supply electrosurgical energy from the power cord 122 to the electrosurgical electrode 128 while (i) the shaft 126 and/or the electrosurgical electrode 128 telescopically moves relative to the handle 124, and/or (ii) the electrosurgical electrode 128 rotates relative to the handle 124.

Although the electrosurgical device 112 includes the user input device(s) 130 in FIG. 1, the user input device(s) 130 can be separate from the electrosurgical device 112 in another example. For instance, the user input device(s) 130 can additionally or alternatively include one or more foot pedals that are actuatable to control operation of the electrosurgical device 112 as described above. The foot pedal(s) can be communicatively coupled to the electrosurgical generator 110 to provide a signal responsive to actuation of the foot pedal(s).

As shown in FIG. 1, in some implementations, the electrosurgical device 112 can additionally include one or more light sources 138 that are configured to emit light. In some examples that include the light source(s) 138, the user input device(s) 130 can be operable to cause the light source(s) 138 to generate light that can be emitted by the electrosurgical device 112 to illuminate an area of interest (e.g., a target tissue at the surgical site). In some implementations, the light source(s) 138 can be located at a distal end of the housing 123 and/or a distal end of the shaft 126 to directly provide light in a distal direction and illuminate a surgical distal of the electrosurgical electrode 128.

In other implementations, as shown in FIG. 1, the light source(s) 138 can be optically coupled to an optical structure 140, which is configured to receive the light emitted by the light source(s) 138 and transmit the light in a distal direction toward a surgical site to illuminate the surgical site while performing electrosurgery using the electrosurgical electrode 128. Although arranging the light source(s) 138 to directly illuminate a surgical field can help, for instance, to reduce a cost of manufacture, transmitting the light using the optical structure 140 can help to improve a quality of light transmitted from the electrosurgical device 112 (e.g., by providing light with improved uniformity and/or reduced heat generation).

As examples, in implementations that include the optical structure 140, the optical structure 140 can include at least one optical structure selected from among a group consisting of an optical lens, a non-fiber optic optical waveguide, and an optical fiber. When the optical structure 140 includes the optical lens (e.g., a parabolic reflector lens, an aspheric lens, and/or a Fresnel lens), the optical structure 140 can help to direct the light emitted by the light source 138 in the distal direction and thereby improve a quality of the light illuminating the surgical site. The optical structure 140 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 140 can additionally or alternatively include other light shaping optical elements such as, for instance, a plurality of facets, one or more prisms, and/or one or more optical gratings. Although the optical structure 140 can help to improve a quality of the light directed to the surgical site, the electrosurgical device 112 can omit the optical structure 140 and instead emit the light from the light source 138 directly to the surgical field without transmitting the light through the optical structure 140 in other examples.

In FIG. 1, the light source 138 can be coupled to the shaft 126. As such, the light source 138 can also move telescopically with the shaft 126 relative to the handle 124. However, in other examples, the light source 138 can be in the interior cavity 145 of the handle 124 and/or coupled to an exterior surface of the handle 124. As examples, the light source 138 can include one or more light emitting diodes (LEDs), organic light emitting diodes (OLEDs), optical fibers, non-fiber optic waveguides, and/or lenses. Additionally, for example, the light source 138 can include a LED printed circuit board having one or more light sources (e.g., LEDs).

The optical structure 140 can be at a distal end of the shaft 126. In some examples, the optical structure 140 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 123 and/or the electrosurgical device 112 relative to the target tissue. However, in other examples, the optical structure 140 can extend partially but not fully around the electrosurgical electrode 128.

In implementations that include the light source 138, the user input device(s) 130, the printed circuit board 132, the switches 136, and/or the housing conductor(s) 134 can additionally supply an electrical power from a direct current (DC) power source 142 to the light source 138. In one example, the DC power source 142 can include a battery disposed in the handle 124, the plug of the power cord 122, and/or a battery receptacle located along the power cord 122 between the handle 124 and the plug. Although the electrosurgical device 112 includes the DC power source 142 in FIG. 1, the DC power source 142 can be separate and distinct from the electrosurgical device 112 in other examples. For instance, in another example, the electrosurgical generator 110 can include the DC power source 142.

Additionally, in implementations that include the light source 138, the user input device(s) 130 can be operable to cause the light source 138 to emit the light. In one example, the user input device(s) 130 can include a button that independently controls the light source 138 separate from the electrode user input device(s) 135 that control the electrosurgical operational modes of the electrosurgical device 112. In another example, the user input device(s) 130 and the printed circuit board 132 can be configured such that operation of the electrode user input device(s) 135 that control the electrosurgical operational mode simultaneously control operation of the light source 138 (e.g., the light source 138 can be automatically actuated to emit light when a button is operated to apply the electrosurgical energy at the electrosurgical electrode 128).

As shown in FIG. 1, responsive to operation of the user input device(s) 130 to actuate the light source 138, the DC power source 142 can supply the electrical power (e.g., a DC voltage) to the light source 138 via the printed circuit board 132 and/or the housing conductor(s) 134. In this implementation, one or more of the conductive elements of the housing conductor(s) 134 can be configured to supply the electrical power from the DC power source 142 to the light source 138 and/or return the electrical power from the light source 138 to the DC power source 142. Accordingly, the housing conductor(s) 134 can additionally or alternatively assist in providing electrical communication between the DC power source 142 and the light source 138 as the shaft 126 and the light source 138 telescopically move relative to the handle 124.

Although the user input device(s) 130 on the handle 124 can be operated to control the operation of the light source 138 in the examples described above, the light source 138 can be additionally or alternatively operated by one or more user input device(s) on the electrosurgical generator 110 (e.g., via the user interface 116) and/or on the plug of the power cord 122.

In some examples, the electrosurgical device 112 can additionally or alternatively include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump 144 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 144 and the electrosurgical generator 110 can be provided as separate devices or integrated in a single device (e.g., in a common housing).

As shown in FIG. 1, the shaft 126 can include a smoke evacuation channel 146 in the inner cavity of the shaft 126. The smoke evacuation channel 146 can also include one or more smoke inlets at one or more positions around the electrosurgical electrode 128. In some examples, the smoke evacuation channel 146 can include a plurality of smoke inlets on a plurality of sides of the electrosurgical electrode 128, and/or one or more smoke inlets extending around at least a portion of a circumference of the electrosurgical electrode 128. In this arrangement, the smoke inlet(s) of the smoke evacuation channel 146 can help to receive surgical smoke into the smoke evacuation channel 146 in a plurality of rotational alignments of the electrosurgical electrode 128 relative to the handle 124 and/or the electrosurgical device 112 relative to the target tissue.

In an example, the smoke evacuation channel 146 of the shaft 126 defines a first portion of a smoke flow path, and the interior cavity 145 of the handle 124 defines a second portion of a smoke flow path. In this arrangement, the surgical smoke can be received from the surgical site into the smoke evacuation channel 146 of the shaft 126, and flow proximally along the smoke evacuation channel 146 to the interior cavity 145 of the handle 124. In the interior cavity 145 of the handle 124, the smoke can further flow to a smoke tube 150 that is coupled to a proximal end of the handle 124 and configured to convey smoke from the handle 124 to the suction pump 144.

As noted above, the electrosurgical electrode 128 can apply the electrosurgical energy to a target tissue to perform an electrosurgical operation (e.g., cutting, coagulating, ablating, and/or sealing the target tissue). Within examples, the electrosurgical electrode 128 can include an electrosurgical substrate formed from an electrically conductive material. As an example, the electrically conductive material can be stainless steel.

The electrosurgical substrate can extend in an axial direction from a proximal end of the electrosurgical electrode 128 to a distal end of the electrosurgical electrode 128. The proximal end of the electrosurgical electrode 128 can receive electrosurgical energy from the electrosurgical device 112 (e.g., via the housing conductor 134 as described above), and a distal working portion of the electrosurgical electrode 128 can apply the electrosurgical energy to the target tissue. In one implementation, the electrosurgical substrate can include a shank portion that extends from the proximal end of electrosurgical electrode 128 to the distal working portion of the electrosurgical electrode 128. The distal working portion can be configured to use the electrosurgical energy to at least one of cut or coagulate tissue in a monopolar electrosurgical operation.

In some examples, the distal working portion can define an electrosurgical blade. For instance, the electrosurgical blade can include (i) a first lateral surface, (ii) a second lateral surface opposite the first lateral surface, (iii) a first major surface extending between the first lateral surface and the second lateral surface on a first side of the electrosurgical blade, and (iv) a second major surface extending between the first lateral surface and the second lateral surface on a second side of the electrosurgical blade that is opposite the first side. The first lateral surface and the second lateral surface have surface areas that are relatively small compared to surface areas of the first major surface and the second major surface such that a thickness (e.g., a dimension between the first major surface and the second major surface) of the electrosurgical blade is relatively small as compared to a length (e.g., a dimension extending between the proximal end and the distal end of the electrosurgical electrode 128) and a width (e.g., a dimension between the first lateral surface and the second lateral surface).

In some examples, the distal working portion of the electrosurgical electrode 128 can also include an outer layer of material covering at least a portion (or an entirety) of the electrosurgical substrate. For instance, the outer layer of material can be formed from at least one material selected from a group consisting of: a polymeric material, a fluorocarbon material (e.g., polytetrafluoroethylene (PTFE)), silicone, enamel, a ceramic material, and inorganic lubricant material (e.g., titanium nitride, zirconium nitride, titanium aluminum nitride, and nitron). The outer layer of material can help to, for example, inhibit eschar build-up and/or focus the electrosurgical energy to one or more portions of the electrosurgical electrode 128.

In some examples, the distal working portion of the electrosurgical electrode 128 can additionally include an intermediate layer between the electrosurgical substrate and the outer layer. The intermediate layer can be configured to provide thermal conductivity to help mitigate heating of the outer layer leading to a breakdown of the outer layer. The intermediate layer can also be configured to maintain the electrical conductivity of the electrosurgical substrate such that the intermediate layer does not degrade the transmission of the electrosurgical energy from the electrosurgical substrate to the target tissue.

The intermediate layer can be an anisotropic thermally conductive material, whereby the in-plane (e.g., parallel to the electrode surface) thermal conductivity substantially exceeds the out-of-plane (e.g., perpendicular to the electrode surface) thermal conductivity. The anisotropic thermally conductive material having a coefficient of thermal expansion matched (or approximately 10% greater or approximately 10% lower) to the electrosurgical substrate and outer layer. As an example, this intermediate layer can include at least one material selected from a group consisting of: pyrolytic graphite/carbon, graphene, and Molybdenum disulfide.

As described above, the electrosurgical device 112 includes one or more features that provide for automated extension and/or retraction of the shaft 126 relative to the handle 124. As shown in FIG. 1, the electrosurgical device 112 includes a linear actuator 152 in the interior cavity 145 of the handle 124. The linear actuator 152 is operable to axially move the shaft 126 relative to the handle 124 (e.g., in the proximal direction and the distal direction along the longitudinal axis of the shaft 126).

In one example, the linear actuator 152 can be an electro-mechanical actuator that can transduce electrical energy into linear displacement of the shaft 126 relative to the handle 124. For instance, in the example shown in FIG. 1, the linear actuator 152 can include a motor 154 that is configured to transduce electrical current into axial movement of the shaft 126. The motor 154 can be electrically coupled to the DC power source 142 and/or the electrosurgical generator 110 (e.g., via the printed circuit board 132) to receive the electrical current for moving the shaft 126. As examples, the motor 154 can be a rotary electric motor and/or a linear motor.

Additionally, as described above, the shaft user input device 133 is configured to control operation of the linear actuator 152 and move the shaft 126 relative to the handle 124. As examples, the shaft user input device 133 can include one or more devices selected from a group consisting of: one or more buttons, one or more rocker switches, one or more sliders, one or more dials, one or more knobs, and one or more touch pads. In one implementation, the at least one electrode user input device 135 and the shaft user input device 133 can be sequentially operable while gripping the handle 124 with a single hand and without removing the single hand from the handle 124 (e.g., the electrode user input device(s) 135 and the shaft user input device(s) 133 can be positioned adjacent to each other and separated by a distance that is within a range of motion of one finger while other fingers fixedly grip the handle 124). This can help to further enhance operational efficiencies and ease of use.

In some examples, the shaft user input device 133 is operable between a first state, a second state, and a third state. When the shaft user input device 133 is in the first state, the linear actuator 152 maintains a position of the shaft 126 relative to handle 124. When the shaft user input device 133 is in the second state, the linear actuator 152 moves the shaft 126 distally relative to the handle 124. When the shaft user input device 133 is in the third state, the linear actuator 152 moves the shaft 126 proximally relative to the handle 124. Additionally, in one implementation, the shaft user input device 133 can be biased toward the first state. This can help to automatically cease moving the shaft 126 and hold the shaft 126 in a selected position relative to the handle 124 responsive to the user ceasing actuation of the shaft user input device 133.

In some examples, the electrosurgical device 112 can be configured to (i) allow the linear actuator 152 to move the shaft 126 relative to the handle 124 only when no electrosurgical energy is supplied to the electrosurgical electrode 128, and (ii) supply electrosurgical energy to the electrosurgical electrode 128 only while the shaft 126 remains axially fixed relative to the handle 124 (e.g., while the shaft 126 is not moving relative to the handle 124). This can enhance safe operation of by preventing inadvertent movement of the shaft 126 while performing electrosurgery and/or preventing inadvertent supply of electrosurgical energy while adjusting a position of the shaft 126 (and, in some examples, the electrosurgical electrode 128) relative to the handle 124.

In one implementation, the printed circuit board 132 can be communicatively coupled with the at least one electrode user input device 135 and the shaft user input device 133, and the printed circuit board 132 is configured to prevent the supply of the electrosurgical energy to the electrosurgical electrode 128 while the shaft user input device 133 is operated to move the shaft 126. Also, in this implementation, the printed circuit board 132 is configured to prevent the linear actuator 152 moving the shaft 126 relative to the handle 124 while the at least one electrode user input device 135 is operated to supply the electrosurgical energy to the electrosurgical electrode 128.

As shown in FIG. 1, in some examples, the electrosurgical device 112 can additionally include a sensor 156 that is configured to sense a parameter and, based on the parameter sensed by the sensor 156, generate a signal indicative of a position of the shaft 126 relative to the handle 124. The printed circuit board 132 can be communicatively coupled to the sensor 156, and the printed circuit board 132 can be configured to: (i) receive the signal from the sensor 156, (ii) based on the signal, make a determination that a fault condition occurred, and (iii) responsive to the determination that the fault condition occurred, cease operation of the linear actuator 152. For instance, example fault conditions can include one or more conditions selected from a group consisting of: (i) a collision between the shaft 126 and/or the electrosurgical electrode 128 and an external object, and (ii) a jam that inhibits or prevents the shaft 126 from moving relative to the handle 124 (e.g., shaft 126 not moving relative to the handle 124 even though current is being provided to the linear actuator 152).

In another example, the sensor 156 and the printed circuit board 132 can be configured to stop the linear actuator 152 when the shaft 126 reaches a proximal end of a range of motion and/or a distal end of the range of motion for the shaft 126 relative to the handle 124. For instance, the printed circuit board 132 can be configured to: (i) receive the signal from the sensor 156, (ii) based on the signal, make a determination that the shaft 126 is at an end of the range of motion (e.g., the proximal end or the distal end of the range of motion), and (iii) responsive to the determination that the shaft 126 is at the end of the range of motion, cease operation of the linear actuator 152. This can help to reduce wear and tear on the linear actuator 152.

In another example, the printed circuit board 132 can be configured to (i) store, based on the signal received from the sensor 156, an indication of a last position of the shaft 126 relative to the handle 124, and (ii) cause the linear actuator 152 to move the shaft 126 to the last position responsive to movement of the shaft 126 relative to the handle 124 during an interruption of electrical power to the electrosurgical device 112. For instance, while the electrosurgical device 112 is unplugged from the electrosurgical generator 110, the shaft 126 may be manually moved relative to the handle 124. In some instances, this may inadvertently occur during handling or while swapping out the electrosurgical electrode 128. Automatically returning to the last stored position prior to the power interruption can help to improve operational efficiencies and enhance ease of use.

As examples, the sensor 156 can additionally or alternatively include one or more sensors selected from a group consisting of: a potentiometric position sensor, an inductive position sensor, an eddy current-based position sensor, a capacitive position sensor, a magnetorestrictive position sensor, a Hall Effect sensor, a fiber-optic position sensor, an optical position sensor, and an ultrasonic positions sensor. In one example, the sensor 156 can be an anisotropic magneto-resistive (AMR) sensor. This may be beneficial for at least the reason that the AMR sensor is a non-contact sensor, which can help to extend useful life of the sensor 156, reduce a total power budget, and/or reduce wear and tear on the sensor 156 (or other components that may otherwise be contacted by the sensor 156). Also, in an example, the parameter can be a current used by the motor 154 of the linear actuator 152 to move the shaft 126.

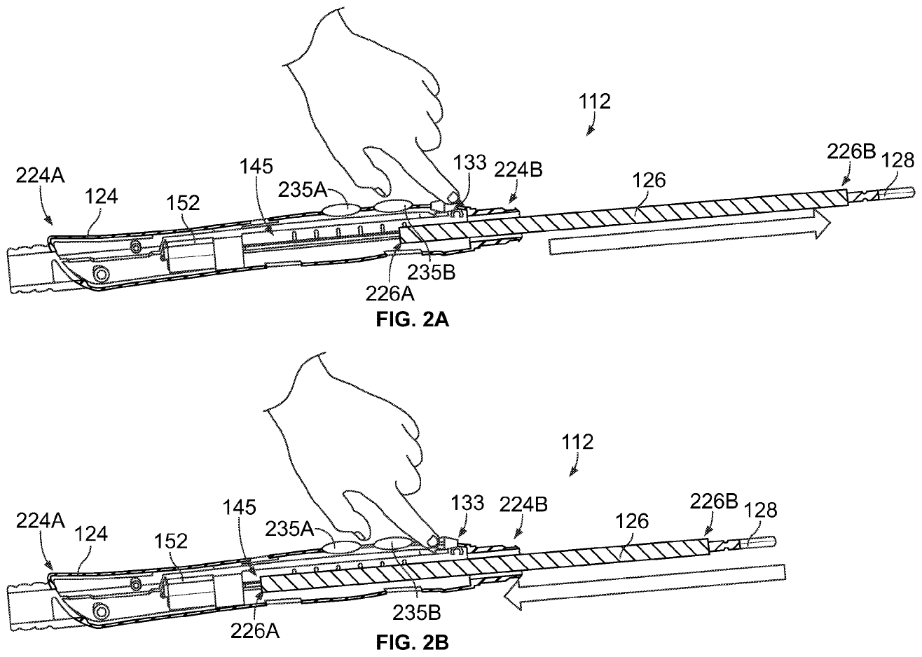

FIGS. 2A-2B depict cross-sectional views of the electrosurgical device 112, according to an example implementation of the electrosurgical device 112 shown in FIG. 1. In particular, FIG. 2A depicts a cross-sectional view taken through a longitudinal axis while the shaft user input device 133 is operated to extend the shaft 126 relative to the handle 124, and FIG. 2B depicts a cross-sectional view taken through the longitudinal axis while the shaft user input device 133 is operated to retract the shaft 126 relative to the handle 124.

As shown in FIGS. 2A-2B, the handle 124 has a proximal end 224A and a distal end 224B, and the handle defines the interior cavity 145. The shaft 126 extends from the distal end 224B of the handle 124. At least a portion of the shaft 126 is in the interior cavity 145 of the handle 124, and the shaft 126 is telescopically movable relative to the handle 124. For instance, at least a proximal end 226A of the shaft 126 is in the interior cavity 145. The electrosurgical electrode 128 extends from a distal end 226B of the shaft 126.

In this example, the electrosurgical device 112 includes two electrode user input devices 235A, 235B that are operable to control the supply of electrosurgical energy to the electrosurgical electrode 128. The electrode user input devices 235A, 235B can be configured to operate the electrosurgical device 112 according to different modes of operation. For instance, operating the electrode user input device 235A can cause the electrosurgical energy to be supplied to the electrosurgical electrode 128 according to a first mode (e.g., the electrosurgical energy can have a first power, a first waveform, a first frequency, etc.) and operating the electrode user input device 235B can cause the electrosurgical energy to be supplied to the electrosurgical electrode 128 according to a second mode (e.g., the electrosurgical energy can have second power, second waveform, second frequency, etc.). Although the electrode user input devices 235A, 235B are shown as buttons in FIGS. 2A-2B, the electrode user input devices 235A, 235B can take different forms in other examples (e.g., the electrode user input devices 235A, 235B can include one or more devices selected from a group consisting of: one or more buttons, one or more rocker switches, one or more sliders, one or more dials, one or more knobs, and one or more touch pads). Additionally, although the electrosurgical device 112 includes two electrode user input devices 235A, 235B in FIGS. 2A-2B, the electrosurgical device 112 can include one or more than two electrode user input devices 235A, 235B in other examples.

Additionally, as shown in FIGS. 2A-2B, the linear actuator 152 is in the interior cavity 145 of the handle 124, and the linear actuator 152 is operable to axially move the shaft 126 relative to the handle 124. In this example, the linear actuator 152 is fixedly disposed in a proximal portion of the handle 124 such that the linear actuator 152 does not move relative to the handle 124 while the linear actuator 152 moves the shaft 126. In one implementation, the handle 124 can include one or more ribs that extend inwardly from an interior wall of the handle 124 to limit or prevent movement of the linear actuator 152 relative to the handle 124.

The shaft user input device 133 configured to control operation of the linear actuator 152 and move the shaft 126 relative to the handle 124. In this example, the shaft user input device 133 includes a rocker switch having the first state, the second state, and the third state described above. However, the shaft user input device 133 can be configured differently in other examples. In the example shown in FIGS. 2A-2B, the electrode user input devices 235A, 235B and the shaft user input device 133 are sequentially operable while gripping the handle 124 with a single hand and without removing the single hand from the handle 124.

Although not shown in FIGS. 2A-2B, when the shaft user input device 133 is not actuated, the shaft user input device 133 is biased to the first state, in which the linear actuator 152 maintains the position of the shaft 126 relative to handle 124. In one example, the shaft user input device 133 can include a biasing member such as, for instance, a spring to bias the rocker switch from the second state to the first state, and from the third state to the first state. As shown in FIG. 2A, when the shaft user input device 133 is in the second state, the linear actuator 152 moves the shaft 126 distally relative to the handle 124. As shown in FIG. 2B, when the shaft user input device 133 is in the third state, the linear actuator 152 moves the shaft 126 proximally relative to the handle 124.

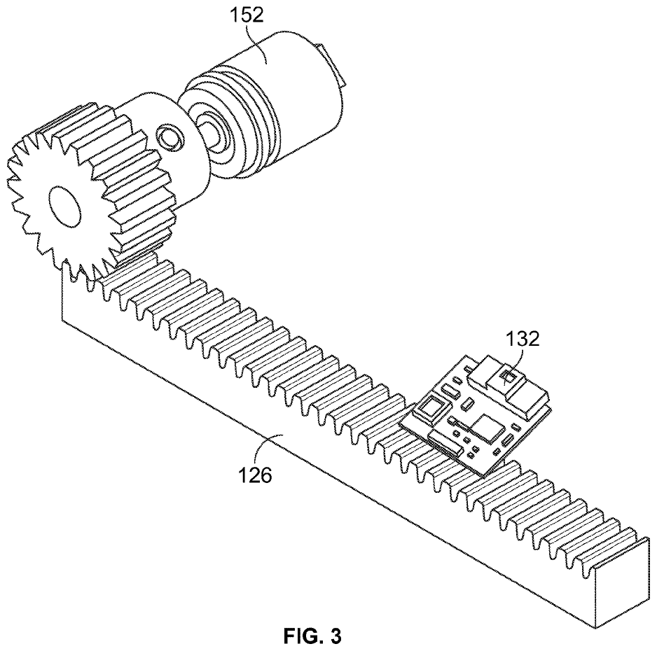

As described above, in some implementations, the electrosurgical device 112 can include the sensor 156. FIG. 3 depicts an assembly of the linear actuator 152, a portion of the shaft 126, and the printed circuit board 132 according to one example. The linear actuator 152, the portion of the shaft 126, and the printed circuit board 132 can be located in the interior cavity 145 of the handle 124 as described above.

The sensor 156 can be configured to sense a parameter and, based on the parameter sensed by the sensor 156, generate a signal indicative of an axial position of the shaft 126 relative to the handle 124. Additionally, in FIG. 3, the printed circuit board 132 can be communicatively coupled to the sensor 156 and configured to receive the signal from the sensor 156. The printed circuit board 132 can be further configured to, based on the signal, make a determination that a fault condition occurred (e.g., a collision occurred and/or a jam occurred). Responsive to the determination that the fault condition occurred, the printed circuit board 132 can cease operation of the linear actuator 152.

Additionally or alternatively, the printed circuit board 132 can store the last position of the shaft 126 relative to the handle 124 and cause the linear actuator 152 to move the shaft 126 to the last position responsive to movement of the shaft 126 relative to the handle 124 during an interruption of electrical power to the electrosurgical device 112.

In an example, the electrosurgical device 112 receives a first input via the shaft user input device 133 and moves, via the linear actuator 152, the shaft 126 axially relative to the handle 124 in response to receiving the first input. For example, the first input is a first type of input received by a distal portion of the shaft user input device 133 and the linear actuator 152 moves the shaft 126 distally relative to the handle 124. Additionally, the shaft user input device 133 receives an input of a second type, for example at a proximal portion of the shaft user input device 133, and the linear actuator 152 moves the shaft 126 proximally relative to the handle 124 in response to receiving the second type of input.

In some examples, the electrode user input device 135, 235A, 235B receives a third input and the electrosurgical generator 110 provides electrical energy to the electrosurgical electrode 128 in response to receiving the third input.

Additionally or alternatively, one or more of the sensors 156 and/or the printed circuit board 132 can sense that the shaft user input device 133 is not being operated (e.g., not receiving input) and enable the electrode user input device 135 in response to the sensing such that receiving input at the electrode user input device 135 causes electrical energy to be provided from the electrosurgical generator 110 to the electrosurgical electrode 128. Thus, in this example, the electrosurgical electrode 128 is enabled only when it is determined that the shaft user input device 133 is not being used to move the shaft 126 relative to the handle 124.

In some examples, one or more of the sensors 156 and/or the printed circuit board 132 senses that the shaft user input device 133 is being operated and disables the electrode user input device 135 in response to sensing that the shaft user input device 133 is being operated such that receiving input at the electrode user input device 135 does not cause electrical energy to be provided from the electrosurgical generator 110 to the electrosurgical electrode 128. Thus, in this example, the electrosurgical electrode 128 is disabled because it is determined that the shaft user input device 133 is currently being used to move the shaft 126 relative to the handle 124.

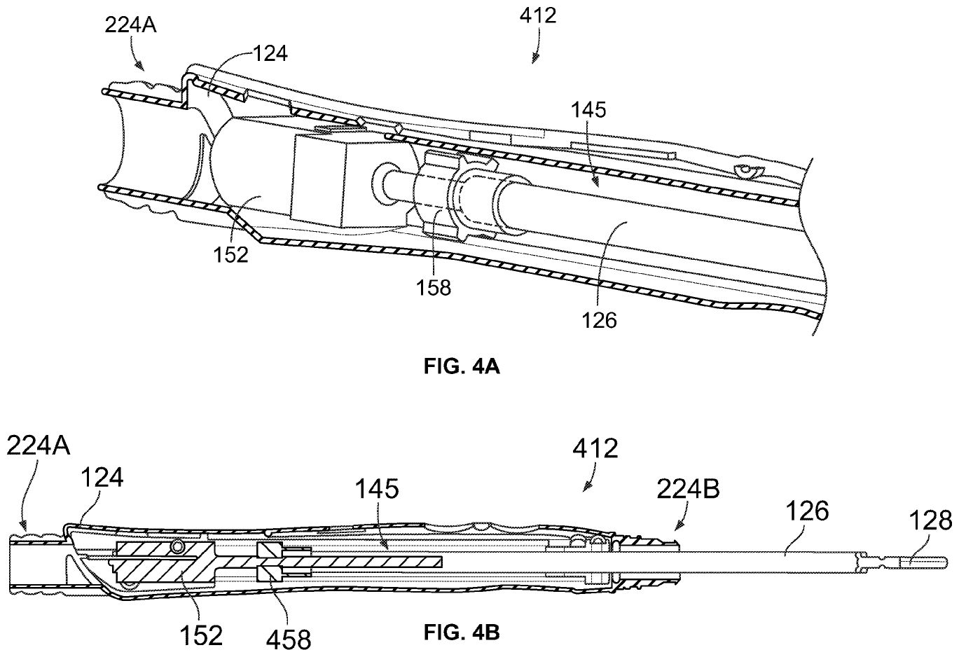

FIG. 4A and FIG. 4B are cross-sectional views of the electrosurgical device 412, according to another example implementation. The electrosurgical device 412 is substantially similar or identical to the electrosurgical device 112 shown and described with respect to FIGS. 1-3, except the electrosurgical device 412 shown in FIGS. 4A-4B includes a gear box 458.

As shown in FIGS. 4A-4B, the gear box 458 can be coupled to the linear actuator 152 and the shaft 126 (e.g., between the linear actuator 152 and the shaft 126). The gear box 458 can include a plurality of gears that can be configured to control a speed at which the shaft 126 moves relative to the handle 124. For instance, the gears of the gear box 458 can have a gear ratio that can receive an input mechanical power from the linear actuator 152 and provide to the shaft 126 an output mechanical power. In some examples, the input mechanical power can be less than the output mechanical power. In other examples, the input mechanical power can be greater than the output mechanical power.

Within some examples, the gear box 458 can additionally or alternatively be configured to reduce backlash when moving the shaft 126 using the linear actuator 152.

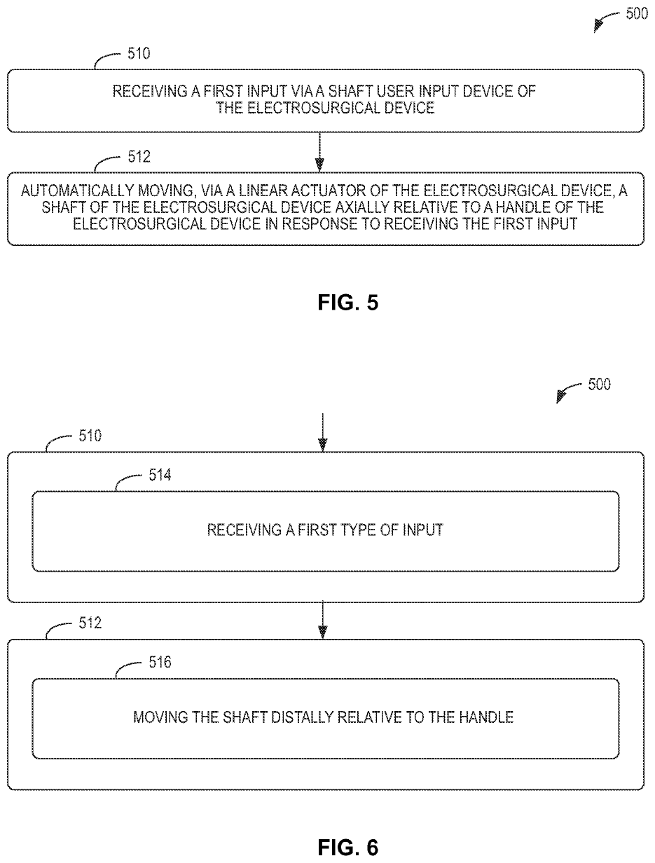

FIG. 5 is a flowchart of a process 500 for operating the electrosurgical device. As shown in FIG. 5, at block 510, the process 500 includes receiving a first input via a shaft user input device of the electrosurgical device. At block 512, the process 500 includes automatically moving, via a linear actuator of the electrosurgical device, a shaft of the electrosurgical device axially relative to a handle of the electrosurgical device in response to receiving the first input. The electrosurgical device can include an electrosurgical electrode extending from a distal end of the shaft.

FIGS. 6-15 depict additional aspects of the process 500 according to further examples. As shown in FIG. 6, receiving the first input at block 510 can include receiving a first type of input at block 514, and automatically moving the shaft at block 512 can include moving the shaft distally relative to the handle at block 516.

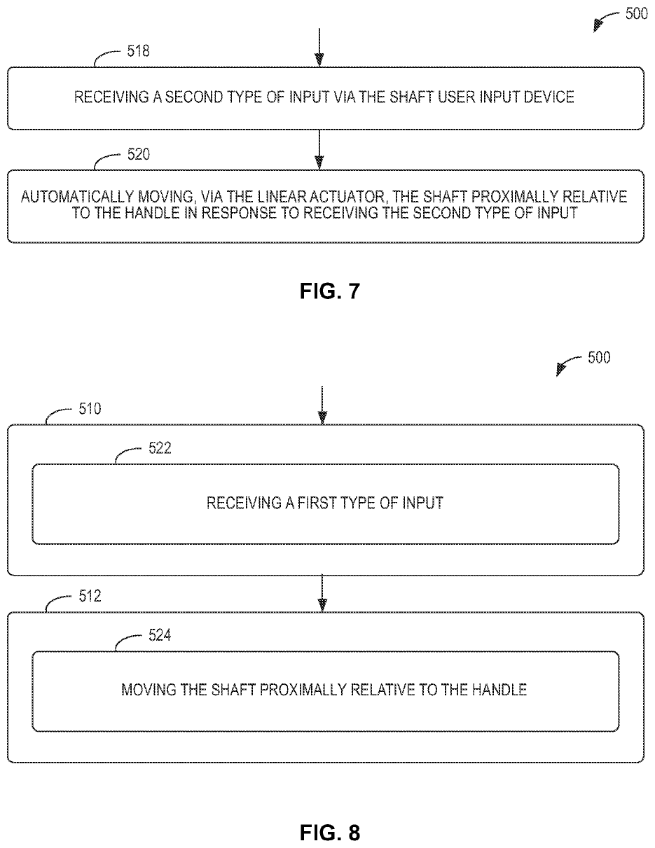

As shown in FIG. 7, the process 500 can also include receiving a second type of input via the shaft user input device at block 518, and automatically moving, via the linear actuator, the shaft proximally relative to the handle in response to receiving the second type of input at block 520.

As shown in FIG. 8, receiving the first input at block 510 can include receiving a first type of input at block 522, and moving the shaft at block 512 can include moving the shaft proximally relative to the handle at block 524.

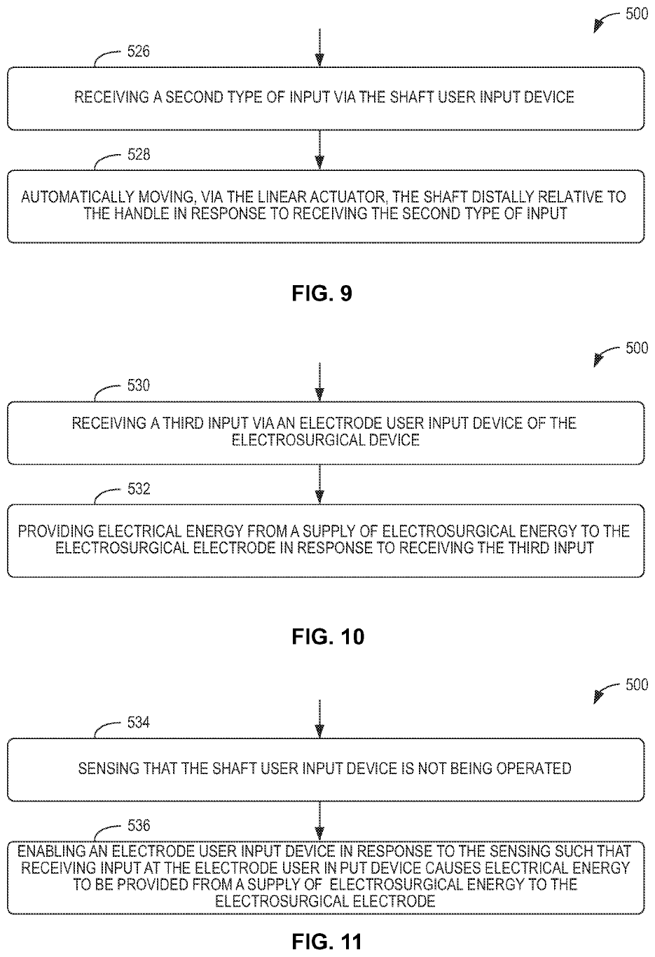

As shown in FIG. 9, the process 500 can also include receiving a second type of input via the shaft user input device at block 526, and automatically moving, via the linear actuator, the shaft distally relative to the handle in response to receiving the second type of input at block 528.

As shown in FIG. 10, the process 500 can also include receiving a third input via an electrode user input device of the electrosurgical device at block 530, and providing electrical energy from a supply of electrosurgical energy to the electrosurgical electrode in response to receiving the third input at block 532.

As shown in FIG. 11, the process 500 can also include sensing that the shaft user input device is not being operated at block 534, and enabling an electrode user input device in response to the sensing such that receiving input at the electrode user input device causes electrical energy to be provided from a supply of electrosurgical energy to the electrosurgical electrode at block 536.

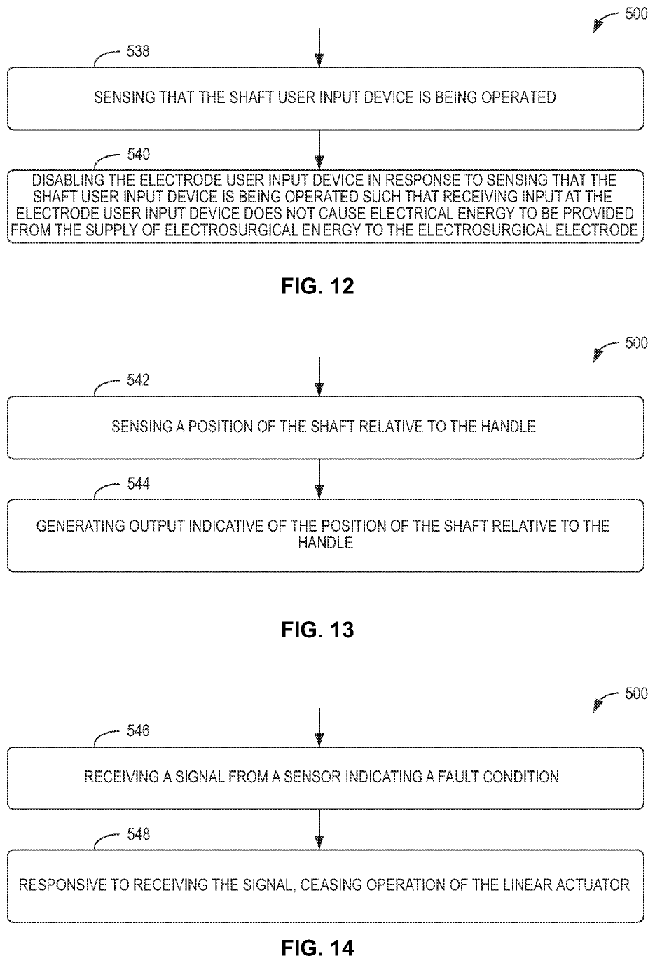

As shown in FIG. 12, the process 500 can also include sensing that the shaft user input device is being operated at block 538, and disabling the electrode user input device in response to sensing that the shaft user input device is being operated such that receiving input at the electrode user input device does not cause electrical energy to be provided from the supply of electrosurgical energy to the electrosurgical electrode at block 540.

As shown in FIG. 13, the process 500 can also include sensing a position of the shaft relative to the handle at block 542, and generating output indicative of the position of the shaft relative to the handle at block 544.

As shown in FIG. 14, the process 500 can also include receiving a signal from a sensor indicating a fault condition at block 546, and responsive to receiving the signal, ceasing operation of the linear actuator at block 548.

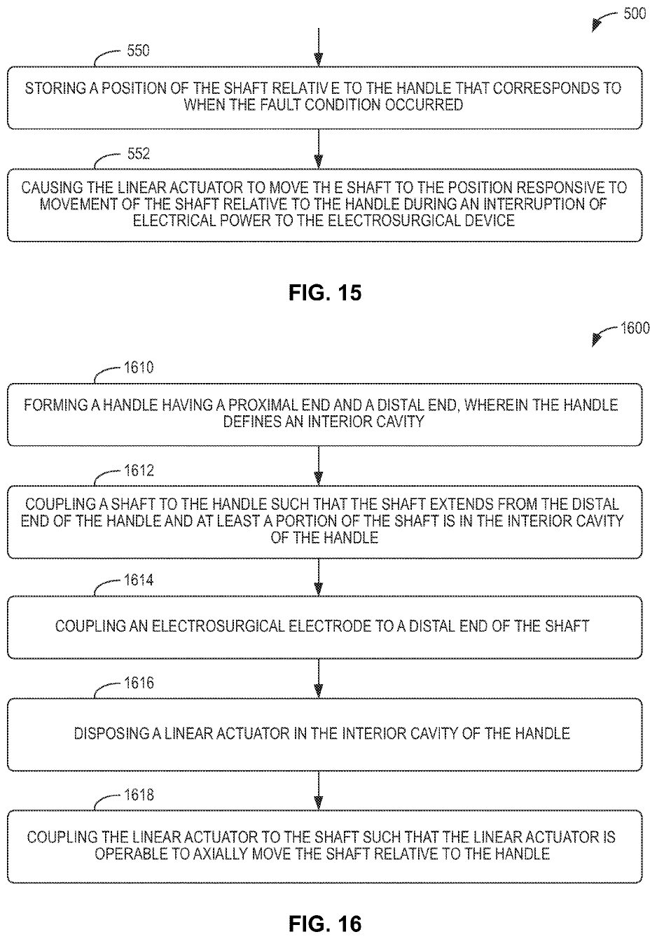

As shown in FIG. 15, the process 500 can also include storing a position of the shaft relative to the handle that corresponds to when the fault condition occurred at block 550, and causing the linear actuator to move the shaft to the position responsive to movement of the shaft relative to the handle during an interruption of electrical power to the electrosurgical device at block 552.

FIG. 16 is a flowchart of a process 1600 for manufacturing an electrosurgical device. At block 1610, the process 1600 includes forming a handle having a proximal end and a distal end, wherein the handle defines an interior cavity. At block 1612, the process 1600 includes coupling a shaft to the handle such that the shaft extends from the distal end of the handle and at least a portion of the shaft is in the interior cavity of the handle. The shaft is telescopically movable relative to the handle. At block 1614, the process 1600 includes coupling an electrosurgical electrode to a distal end of the shaft. At block 1616, the process 1600 includes disposing a linear actuator in the interior cavity of the handle. At block 1618, the process 1600 includes coupling the linear actuator to the shaft such that the linear actuator is operable to axially move the shaft relative to the handle.

The example implementations described above with respect to FIGS. 1-16 include a linear actuator that can be commanded via an electric signal to move the shaft 126 and the electrosurgical electrode 128. In other example implementations, the shaft 126 and the electrosurgical electrode 128 can be moved manually, thereby reducing complexity and cost.

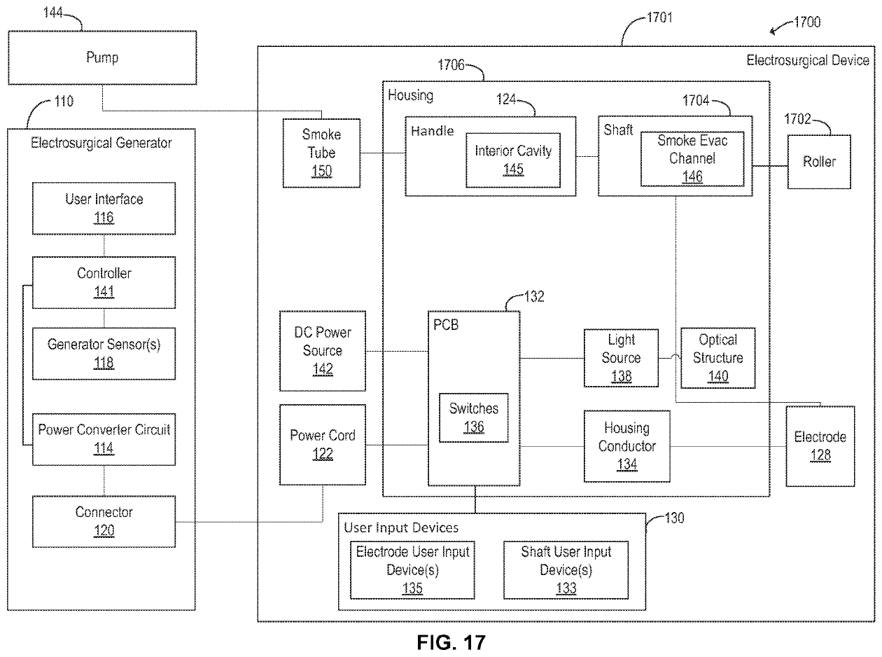

FIG. 17 is a block diagram of an electrosurgical system 1700, according to an example. The electrosurgical system 1700 is similar to the electrosurgical system 100 and identical components are labeled with the same reference numbers.

The electrosurgical system 1700 includes the electrosurgical generator 110 and an electrosurgical device 1701. The electrosurgical device 1701 is similar to the electrosurgical device 112. However, rather than using the linear actuator 152 and the motor 154 to cause linear movement (e.g., extension and retraction) of the electrosurgical electrode 128, the electrosurgical device 1701 includes a roller 1702 that is configured to interact with a shaft 1704. The roller 1702 is configured to be exposed or protruding, at least partially, from a housing 1706 of the electrosurgical device 1701 such that an operator has access to the roller 1702, and is able to manipulate (e.g., rotate) the roller 1702.

The roller 1702 engages (directly or indirectly) the shaft 1704. Particularly, the roller 1702 is configured to interact with the shaft 1704, such that rotation of the roller 1702 by an operator, causes the shaft 1704 and the electrosurgical electrode 128 coupled thereto to move linearly.

In an example, the roller 1702 can be formed as a wheel that interacts with, at least a portion, of the shaft 1704. In one example, the wheel can be formed as a gear (e.g., an involute gear), and the shaft 1704 may have a rack with teeth formed thereon. The gear teeth of the roller 1702 engage with the teeth of the rack of the shaft 1704.

As such, in this example, the roller 1702 and the shaft 1704 form a rack and pinion arrangement, where the shaft 126 operate as the rack, and the roller 1702 as the pinion. With this configuration, rotation of the roller 1702 causes the shaft 1704 to move linearly.

In another example, the roller 1702 may be frictionally engaged with the shaft 1704. In other words, the roller 1702 may have rough surface or a surface having a light adhesive applied thereto. The shaft 1704 may have a portion thereof with a rough surface or with a respective light adhesive applied thereto. This way, rotation of the roller 1702 causes the shaft 1704 to move linearly.

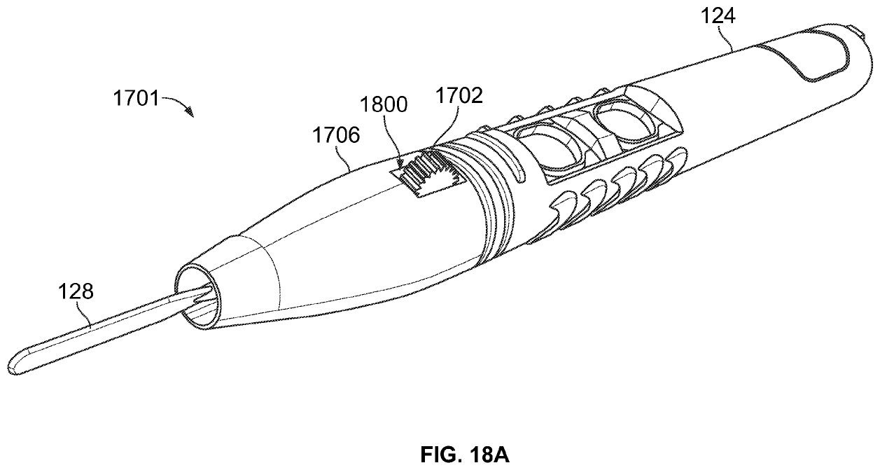

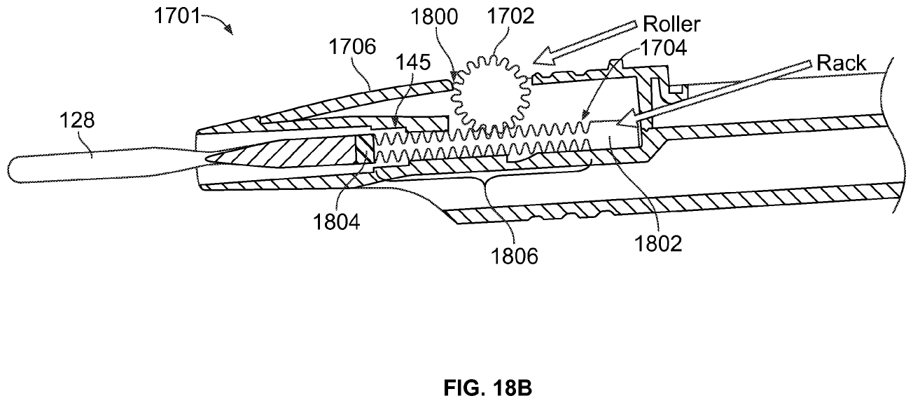

FIG. 18A illustrates a perspective view of the electrosurgical device 1701, and FIG. 18B illustrates a partial cross-sectional view of the electrosurgical device 1701, according to an example. FIGS. 18A, 18B are described together.

As depicted, the housing 1706 has a window or slit 1800. The roller 1702 is disposed partially within the interior cavity 145 of the housing 1706, and protrudes outward through the slit 1800 to be accessible by an operator handling the electrosurgical device 1701 from outside the housing 1706. In the example implementation of FIGS. 18A-18B, the roller 1702 is formed as a gear have involute gear teeth as an example.

Similar to the shaft 126, the shaft 1704 is disposed within the interior cavity 145 of the housing 1706. The shaft 1704 has a proximal end or base 1802. The base 1802 can be generally cylindrical and may have a smooth outer surface.

The shaft 1704 also has a distal end or head 1804. The head 1804 can be generally cylindrical and may have a smooth outer surface.

The shaft 1704 further includes a rack portion 1806 interposed between the base 1802 and the head 1804. As depicted, the rack portion 1806 has teeth formed thereon. Particularly, in the example implementation of FIGS. 18A-18B, the teeth of the rack portion 1806 can be circumferential teeth that may span substantially the entire circumference of the rack portion 1806. In other words, each of the teeth of the rack portion 1806 can be formed a disk or thread formed the shaft 1704.

This configuration ensures that the teeth of the rack portion 1806 engage the teeth of the roller 1702 regardless of the rotational position of the shaft 1704. Thus, in the example implementations where the shaft 1704 is rotatable to orient the electrosurgical electrode 128 as desired, the rack portion 1806 maintains its engagement with the roller 1702 to facilitate linear motion of the shaft 1704 regardless of the rotary position of the shaft 1704.

However, in other example implementations, the teeth of the rack portion 1806 may protrude in one direction only, rather than spanning the entire circumference of the rack portion 1806. For instance, if the shaft 1704 is not rotatable, then continual engagement of the roller 1702 with the rack portion 1806 can be achieved with teeth protruding toward the roller 1702 only, rather than in all directions. In other examples, rather than teeth, the rack portion 1806 may be serrated such that as the roller 1702 rotates, interaction with the serrations of the rack portion 1806 causes the shaft 1704 to move linearly.

In operation, the operator has access to the roller 1702, which protrudes outward through the slit 1800 of the housing 1706. Thus, if the operator rotates the roller 1702 with a finger, the roller 1702 causes the shaft 1704 to move linearly within the interior cavity 145 of the housing 1706. As a result, the linear position of the electrosurgical electrode 128 can be adjusted linearly as desired by the operator. With this configuration, an axis around which the roller 1702 rotates is perpendicular to a longitudinal axis along which the shaft 1704 and the electrosurgical electrode 128 move linearly.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

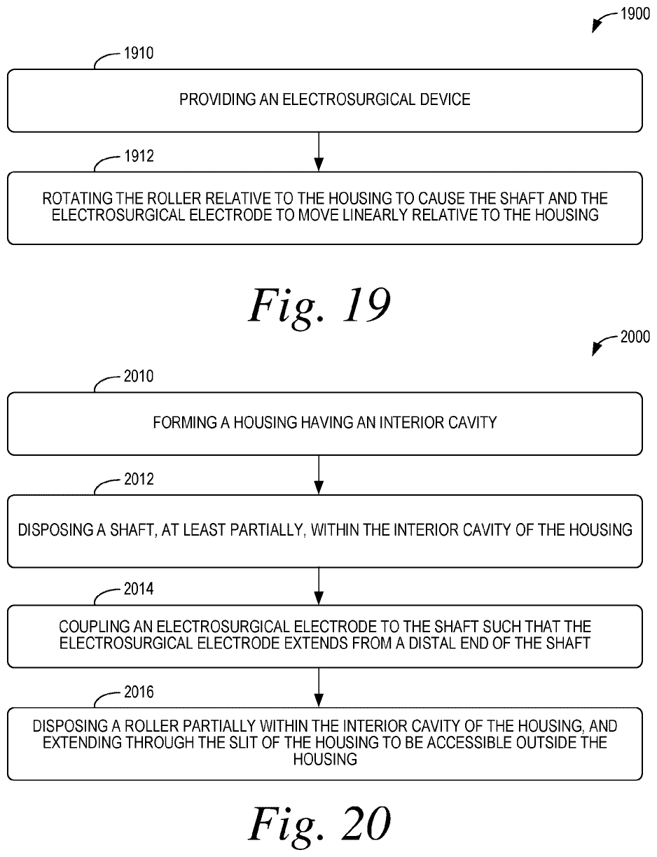

Referring now to FIG. 19, a flowchart for a method 1900 of operating an electrosurgical device shown according to an example. At block 1910, the method 1900 includes providing an electrosurgical device. The electrosurgical device includes a housing having an interior cavity, and the housing has a slit. The electrosurgical device also includes (i) a shaft disposed, at least partially, within the interior cavity of the housing, (ii) an electrosurgical electrode extending from a distal end of the shaft, and (iii) a roller disposed partially within the interior cavity of the housing, and extending through the slit of the housing to be accessible outside the housing. The roller engages the shaft, such that rotation of the roller causes the shaft and the electrosurgical electrode to move linearly relative to the housing. At block 1912, the method 1900 also includes rotating the roller relative to the housing to cause the shaft and the electrosurgical electrode to move linearly relative to the housing.

Referring now to FIG. 20, a flowchart for a method 2000 of manufacturing an electrosurgical device is shown according to an example. At block 2010, the method 2000 includes forming a housing having an interior cavity. The housing has a slit. At block 2012, the method 2000 includes disposing a shaft, at least partially, within the interior cavity of the housing. At block 2014, the method 2000 includes coupling an electrosurgical electrode to the shaft such that the electrosurgical electrode extends from a distal end of the shaft. At block 2016, the method 2000 includes disposing a roller partially within the interior cavity of the housing, and extending through the slit of the housing to be accessible outside the housing. The roller engages the shaft, such that rotation of the roller causes the shaft and the electrosurgical electrode to move linearly relative to the housing.

Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present application is not to be limited by the subject specification, but rather only by the plain meaning of the claim terms employed.

What is claimed is:

1. An electrosurgical device, comprising:
a handle having a proximal end and a distal end, wherein the handle defines an interior cavity;
a shaft extending from the distal end of the handle, wherein at least a portion of the shaft is in the interior cavity of the handle, wherein the shaft is telescopically movable relative to the handle;
an electrosurgical electrode extending from a distal end of the shaft; and
a linear actuator in the interior cavity of the handle, wherein the linear actuator is operable to axially move the shaft relative to the handle.

2. The electrosurgical device of claim 1, wherein the linear actuator comprises a motor that is configured to transduce electrical current into axial movement of the shaft.

3. The electrosurgical device of claim 1, further comprising an electrode user input device and a shaft user input device,
wherein the electrode user input device is operable to control a supply of electrosurgical energy to the electrosurgical electrode, and
wherein the shaft user input device is configured to control operation of the linear actuator and move the shaft relative to the handle.

4. The electrosurgical device of claim 3, wherein the shaft user input device is operable between a first state, a second state, and a third state,
wherein, when the shaft user input device is in the first state, the linear actuator maintains a position of the shaft relative to handle,
wherein, when the shaft user input device is in the second state, the linear actuator moves the shaft distally relative to the handle, and
wherein, when the shaft user input device is in the third state, the linear actuator moves the shaft proximally relative to the handle.

5. The electrosurgical device of claim 4, wherein the shaft user input device is biased toward the first state.

6. The electrosurgical device of any one of claims 3-5, wherein the electrode user input device and the shaft user input device are sequentially operable while gripping the handle with a single hand and without removing the single hand from the handle.

7. The electrosurgical device of any one of claims 3-6, further comprising a printed circuit board communicatively coupled with the electrode user input device and the shaft user input device,
wherein the printed circuit board is configured to prevent the supply of the electrosurgical energy to the electrosurgical electrode while the shaft user input device is operated to move the shaft.

8. The electrosurgical device of claim 7, wherein the printed circuit board is configured to prevent the linear actuator moving the shaft relative to the handle while the electrode user input device is operated to supply the electrosurgical energy to the electrosurgical electrode.

9. The electrosurgical device of any one of claims 1-6, further comprising a sensor configured to sense a parameter and, based on the parameter sensed by the sensor, generate a signal indicative of a position of the shaft relative to the handle.

10. The electrosurgical device of claim 9, further comprising a printed circuit board communicatively coupled to the sensor,
wherein the printed circuit board is configured to:
receive the signal from the sensor and,
based on the signal, make a determination that a fault condition occurred, and
responsive to the determination that the fault condition occurred, cease operation of the linear actuator.

11. The electrosurgical device of claim 10, wherein the sensor is an anisotropic magneto-resistive (AMR) sensor.

12. The electrosurgical device of claim 10, wherein the parameter is a current used by a motor of the linear actuator to move the shaft.

13. The electrosurgical device of any one of claims 10-12, wherein the printed circuit board is configured to:
store, based on the signal, an indication of a last position of the shaft relative to the handle, and
cause the linear actuator to move the shaft to the last position responsive to movement of the shaft relative to the handle during an interruption of electrical power to the electrosurgical device.

14. A method of operating an electrosurgical device, the method comprising:
receiving a first input via a shaft user input device of the electrosurgical device; and
automatically moving, via a linear actuator of the electrosurgical device, a shaft of the electrosurgical device axially relative to a handle of the electrosurgical device in response to receiving the first input, wherein the electrosurgical device includes an electrosurgical electrode extending from a distal end of the shaft.

15. The method of claim 14, wherein receiving the first input comprises receiving a first type of input, and wherein automatically moving the shaft comprises moving the shaft distally relative to the handle.

16. The method of claim 15, further comprising:
receiving a second type of input via the shaft user input device; and
automatically moving, via the linear actuator, the shaft proximally relative to the handle in response to receiving the second type of input.

17. The method of claim 14, wherein receiving the first input comprises receiving a first type of input, and wherein moving the shaft comprises moving the shaft proximally relative to the handle.

18. The method of claim 17, further comprising:
receiving a second type of input via the shaft user input device; and
automatically moving, via the linear actuator, the shaft distally relative to the handle in response to receiving the second type of input.

19. The method of any of claims 14-18, further comprising:
receiving a third input via an electrode user input device of the electrosurgical device; and
providing electrical energy from a supply of electrosurgical energy to the electrosurgical electrode in response to receiving the third input.

20. The method of any of claims 14-19, further comprising:
sensing that the shaft user input device is not being operated; and
enabling an electrode user input device in response to the sensing such that receiving input at the electrode user input device causes electrical energy to be provided from a supply of electrosurgical energy to the electrosurgical electrode.

21. The method of claim 20, further comprising:
sensing that the shaft user input device is being operated; and
disabling the electrode user input device in response to sensing that the shaft user input device is being operated such that receiving input at the electrode user input device does not cause electrical energy to be provided from the supply of electrosurgical energy to the electrosurgical electrode.

22. The method of any of claims 14-21, further comprising:
sensing a position of the shaft relative to the handle; and
generating output indicative of the position of the shaft relative to the handle.

23. The method of any of claims 14-22, further comprising:
receiving a signal from a sensor indicating a fault condition; and
responsive to receiving the signal, ceasing operation of the linear actuator.

24. The method of claim 23, further comprising:
storing a position of the shaft relative to the handle that corresponds to when the fault condition occurred; and
causing the linear actuator to move the shaft to the position responsive to movement of the shaft relative to the handle during an interruption of electrical power to the electrosurgical device.

25. A method of manufacturing an electrosurgical device, the method comprising:
forming a handle having a proximal end and a distal end, wherein the handle defines an interior cavity;
coupling a shaft to the handle such that the shaft extends from the distal end of the handle and at least a portion of the shaft is in the interior cavity of the handle, wherein the shaft is telescopically movable relative to the handle;
coupling an electrosurgical electrode to a distal end of the shaft;
disposing a linear actuator in the interior cavity of the handle; and
coupling the linear actuator to the shaft such that the linear actuator is operable to axially move the shaft relative to the handle.

26. An electrosurgical device comprising:
a housing having an interior cavity, wherein the housing has a slit;
a shaft disposed, at least partially, within the interior cavity of the housing;
an electrosurgical electrode extending from a distal end of the shaft; and
a roller disposed partially within the interior cavity of the housing, and extending through the slit of the housing to be accessible outside the housing, wherein the roller engages the shaft, such that rotation of the roller causes the shaft and the electrosurgical electrode to move linearly relative to the housing.

27. The electrosurgical device of claim 26, wherein an axis around which the roller rotates is perpendicular to a longitudinal axis along which the shaft and the electrosurgical electrode move linearly.

28. The electrosurgical device of any of claims 26-27, wherein the shaft comprises a rack portion having teeth formed thereon, and wherein the roller is formed as a gear having respective teeth engaging with the teeth of the rack portion.

29. The electrosurgical device of claim 28, wherein the teeth of the rack portion are configured as circumferential teeth that span substantially an entire circumference of the rack portion, thereby maintaining engagement between the roller and the rack portion regardless of a rotary position of the shaft.

30. The electrosurgical device of any of claims 26-29, wherein the shaft has serrations that engage with the roller to facilitate linear movement of the shaft upon rotation of the roller.

31. The electrosurgical device of any of claims 26-30, wherein the roller is frictionally engaged with the shaft to facilitate linear movement of the shaft upon rotation of the roller.

32. A method of operating an electrosurgical device, the method comprising:
providing an electrosurgical device comprising:
a housing having an interior cavity, wherein the housing has a slit,
a shaft disposed, at least partially, within the interior cavity of the housing,
an electrosurgical electrode extending from a distal end of the shaft, and
a roller disposed partially within the interior cavity of the housing, and extending through the slit of the housing to be accessible outside the housing,
wherein the roller engages the shaft, such that rotation of the roller causes the shaft and the electrosurgical electrode to move linearly relative to the housing; and
rotating the roller relative to the housing to cause the shaft and the electrosurgical electrode to move linearly relative to the housing.

33. A method of manufacturing an electrosurgical device, the method comprising:
forming a housing having an interior cavity, wherein the housing has a slit;
disposing a shaft, at least partially, within the interior cavity of the housing;
coupling an electrosurgical electrode to the shaft such that the electrosurgical electrode extends from a distal end of the shaft; and
disposing a roller partially within the interior cavity of the housing, and extending through the slit of the housing to be accessible outside the housing,
wherein the roller engages the shaft, such that rotation of the roller causes the shaft and the electrosurgical electrode to move linearly relative to the housing.
